(19) United States
(12) Patent Application Publication
(10) Pub. No.: US 2011/0276004 A1
(43) Pub. Date: Nov. 10, 2011

(54) MICRONEEDLE CAP SYSTEM ENABLING ADJUSTMENT OF OUTFLOW RATE OF ACTIVE SUBSTANCE

(75) Inventors: Boo Joon Sul, Daejeon (KR); Man Hee Han, Daejeon (KR)

(73) Assignee: MITI SYSTEMS INC., Daejeon (KR)

(21) Appl. No.: 13/186,036

(22) Filed: Jul. 19, 2011

Related U.S. Application Data
(63) Continuation of application No. PCT/KR2010/003945, filed on Jun. 18, 2010.

(30) Foreign Application Priority Data
Apr. 15, 2010 (KR) 10-2010-34850

(51) Int. Cl. A61M 5/00

(52) U.S. Cl. 604/173

(57) ABSTRACT

The present invention relates to a microneedle cap system to be mounted to a container containing an active substance. The system comprises a container cap unit and a microneedle unit, which, among others, allows the active substance to be delivered in a desired amount.

### CROSS-REFERENCES TO RELATED APPLICATION

This is a continuation of International Application No. PCT/KR2010/003945, with an international filing date of Jun. 18, 2010, which claims the benefit of Korean Application No. 10-2010-34850 filed on Apr. 15, 2010, the entire contents of which applications are incorporated herein by reference.

### BACKGROUND OF THE DISCLOSURE

1. Technical Field

The present invention relates to a microneedle cap system which comprises a container having an active substance contained therein, a microneedle unit serving to help penetration of an active substance into the skin, and means for adjusting the outflow rate of the active substance. The system allows the active substance and microneedles to be concurrently applied to the skin and the outflow rate of the active substance to be simply and easily adjusted depending on the kind of the active substance and the need of a user, thereby enhancing transdermal absorption efficiency of the active substance.

2. Related Art

An active substance such as a drug or a cosmetic agent/ingredient can be percutaneously administered by, e.g., applying the active substance on a skin of a subject in need of the active substance or attaching a pack, a patch or the like containing the active substance to the skin. These methods enable a sustained drug delivery, are painless, and are simple and convenient in use. They, however, provide low transdermal absorption of the active substance since the stratum corneum, which is an outermost layer of the epidermis of the skin and is 10-60 μm in depth, inhibits the penetration of the active substance into the human body, which is the case particularly when the active substance is hydrophilic or has a large molecular weight.

Alternatively, an active substance can be administered into a subject through a syringe. Conventional syringe needles have a diameter measured in millimeter units (mm) and a length measured in centimeter units (cm). Such syringe needles may stimulate a plurality of pain spots widely distributed in the skin of the subject and cause a considerable pain to the subject. In addition, although this syringe-based method is readily available in hospitals or professional skin care agencies, it is not in general homes.

To address these problems, microneedles having a diameter of several tens to a few hundreds of micrometers (μm) and a length of several tens to a few thousands of micrometers (μm) have been proposed. Since the microneedles are relatively small in diameter and length as compared to the conventional syringe needles, they may stimulate less number of pain spots, thereby causing less pain given to a subject. In addition, by penetrating into and stimulating the epidermal layer of the subject, the microneedles can help healing a burn or an acne scar. Also, the microneedles may induce the production of collagen, thereby contributing to improve skin tone and prevent skin aging. Besides, the microneedles are excellent in in vivo permeability or sustained delivery of active substances. They thus are expected to be used as a new system for delivery of active substances (e.g., a drug, a cosmetic ingredient, etc.).

U.S. Pat. No. 3,964,482 discloses a drug delivery system which includes a microneedle unit configured to puncture the skin of a subject and a drug reservoir formed integrally with the microneedle unit and configured to contain a drug to be delivered to the skin. The system, however, has a drawback in that the drug contained in the drug reservoir may be leaked to the outside during the distribution and storage of the system, requiring a separate device to prevent the leakage.

U.S. Pat. No. 6,537,242 discloses a microneedle system including hollow microneedles coupled to a container such as, for example, a syringe. The microneedle system, however, has a complicated structure and is inconvenient to use. Moreover, mass-production of the hollow microneedles is not easy.

Korean Patent No. 0753872 discloses a microneedle roller system that has a relatively simple structure and can be manufactured relatively easily. The system, however, requires a two step process: applying the microneedle and then applying a drug on the skin, or applying the drug on the skin and then applying the microneedle, thereby causing an inconvenience of use.

U.S. Patent Application Publication No. 2006/0051404 discloses a microstructure which includes hollow microneedles and a microneedle roller. However, mass-production of the hollow microneedles is not easy and making the microneedle roller on a disposable basis increases overall manufacturing cost. Also, repeated use of the microneedle roller may contaminate the drug.

Korean Patent No. 0873642 discloses a method in which an ample is applied to a microneedle roller so that an active substance is injected to a roller head or the skin of a subject. However, the structure of the roller system is relatively complicated, causing manufacturing cost to be increased. Also, the drug can be contaminated by repeated use of the microneedle roller.

In an attempt to solve these problems, the present inventors developed a microneedle cap system including a container that can contain an active substance and a microneedle cap that is attached to the container and has microneedles disposed on a surface thereof, as disclosed in Korean Patent Application No. 2009-0004589. FIG. 1 is a conceptual diagram of the microneedle cap system. When the microneedle cap attached to the container is tapped on the skin of a subject, the microneedles can perforate the stratum corneum and the epidermis layers of the skin to thereby create micropores in the skin. The active substance contained in the container flows out of the container through through-holes formed in the microneedle cap and is applied on the skin. The active substance applied on the skin is then delivered into the skin through the micropores created by the microneedles. However, the size of the through-holes is fixed. Thus, in the case where viscosity of the active substance (or a carrier thereof) contained in the container is low and the through-holes are large relatively, the active substance may flow out in a larger amount than what is needed. On the contrary, in the case where viscosity of the active substance is high and the through-holes are small relatively, the active substance may flow out in a smaller amount than what is needed. Also, various models of microneedle cap systems which have different sizes of the through-holes suited to the property of different active substances are required, which may increase the overall manufacturing costs. Further, the problem of leakage of the active substance may still exist.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in order to solve the above-described problems occurring in the prior art, and it is one of the objects of the present invention to provide a microneedle cap system that allows outflow rate of an active substance to be delivered to be adjusted. Another object of the present invention is to provide a microneedle cap system which can be mass-produced cost-effectively and.

To achieve the above objects, one aspect of the present invention provides a microneedle cap system to be mounted to a container containing an active substance, which comprises a container cap unit and a microneedle unit. The container cap unit is configured to be engaged with the container and includes one or more first through-holes formed on the top surface thereof through which the active substance contained in the container can pass.

The microneedle unit is configured to be rotatably or detachably engaged with the container cap unit and includes a microneedle array formed on the top surface thereof and one or more second through-holes formed therein.

The first through-hole or through-holes can be overlapped with the second through-hole or through-holes depending on the relative position of the microneedle unit with respect to the container cap unit. The area of the region created by the units overlapped can be changed by changing the relative position of the microneedle unit with respect to the container cap unit.

With the system, the active substance can be delivered more easily and the amount of the active substance to be delivered can be conveniently adjusted. In addition, The system can be manufactured and more massively and cost-effectively.

The above and other aspects, features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 represents a conceptual diagram showing a conventional microneedle cap system.

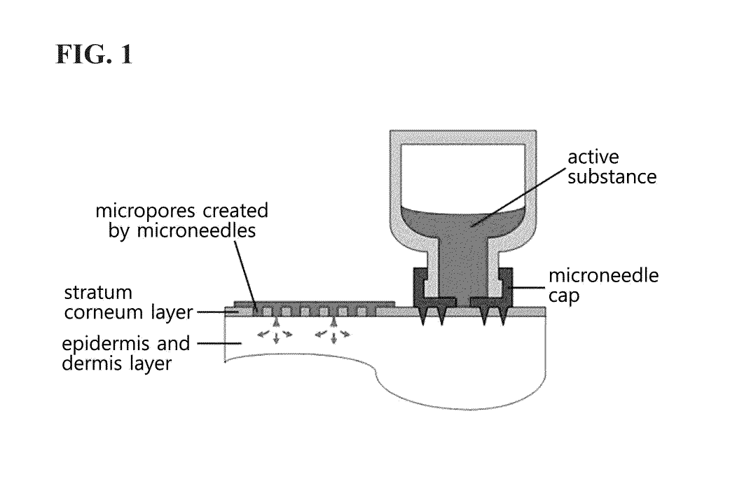

FIG. 2 represents an exploded perspective view and an assembled cross-sectional perspective view of a microneedle cap system according to one embodiment of the present invention;

FIG. 3 represents a perspective and top plan view and a perspective view of a microneedle cap system according to another embodiment of the present invention;

FIG. 4 represents a perspective view of a microneedle cap system according to still another embodiment of the present invention;

FIG. 5 represents an exploded perspective view and an assembled cross-sectional view of a microneedle cap system according to still yet another embodiment of the present invention;

FIG. 6 represents a perspective view of a microneedle cap system having guide grooves formed thereon according to one embodiment of the present invention;

FIG. 7 represents exploded perspective views of a microneedle cap system according to a further embodiment of the present invention;

FIG. 8 represents an exploded perspective view and an assembled cross-sectional perspective view of a microneedle cap system according to a still further embodiment of the present invention; and

FIG. 9 represents a perspective view of a microneedle cap system according to a still yet further embodiment of the present invention.

### DETAILED DESCRIPTION OF EMBODIMENTS

Hereinafter, the present invention will be described in detail in connection with the preferred embodiments with reference to the accompanying drawings. However, the embodiments of the microneedle cap systems shown in the drawings are merely for illustrative purposes, and are not intended to limit or modify the spirit and scope of the invention as above described and illustrated. Also, it will be understood by those skilled in the art that various modifications and variations can be made to the present invention without departing from the spirit and scope of the appended claims based on the illustrative embodiments.

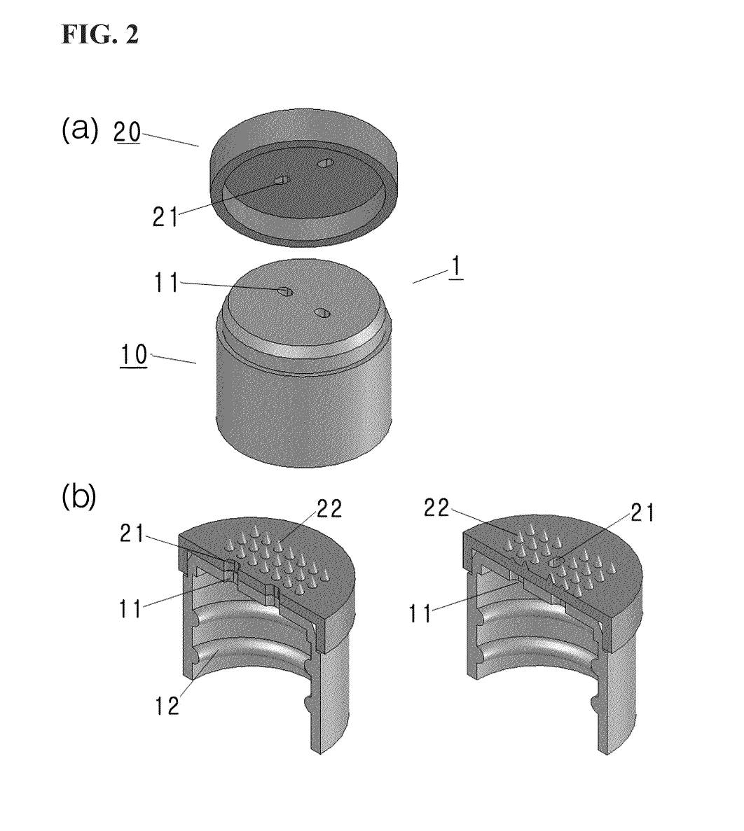

FIG. 2(A) is an exploded perspective view of a microneedle cap system according to an embodiment of the present invention, and FIG. 2(b) is an assembled cross-sectional perspective view of the microneedle cap system. As shown in FIGS. 2(a) and 2(b), the microneedle cap system includes a container cap unit 10 and a microneedle unit 20. The container cap unit 10 is configured to be engaged with at least a part of a container (e.g., a vial) containing an active substance. The container cap unit 10 has at lease one first through-hole 11 formed on the top surface thereof so as to allow the active substance contained in the container to pass therethrough.

The microneedle unit 20 includes a microneedle array 22 formed on the top surface thereof. The microneedle unit 20 is configured to be engaged with the container cap unit 10 in such a fashion that the bottom surface of the microneedle unit 20 is in close contact with the top surface of the container cap unit so as to prevent the active substance contained in the container from leaking between the container cap unit 10 and the microneedle unit 20.

The microneedle unit 20 includes at lease one second through-hole 21 at position(s) corresponding to the position(s) of the first through-hole(s) 11. The outflow rate of the active substance can be adjusted by changing the relative positions of the microneedle unit 20 and the container cap unit 10, For example, the outflow rate can be adjusted by rotating the microneedle unit 20 in a state in which the microneedle unit 20 is engaged with the container cap unit 10. It also can be adjusted by disengaging the microneedle unit 20 from the container cap unit 10, rotating the container cap unit 10, the microneedle unit 20, or both, and then re-engaging the microneedle unit 20 with the container cap unit 10.

As shown in the left side of FIG. 2 (b), when the first through-hole 11 and the second through-hole 21 are juxtaposed with each other, the active substance may flow out of the container. Although not shown in the drawings, when the first through-hole 11 and the second through-hole 21 are overlapped to some extent, a smaller amount of the active substance may flow out of the container. On the other hand, as shown in the right side of FIG. 2 (b), when the first through-hole 11 and the second through-hole 21 are disposed in such a fashion as not to be overlapped with each other, the active substance may not flow out of the container. As shown above, by adjusting the overlapped area between the first through-hole 11 and the second through-hole 21, a user can conveniently adjust the outflow rate of the active substance taking into consideration of the viscosity of the active substance and other conditions/needs.

Although it is shown in FIG. 2 that the container cap unit 10 and the microneedle unit 20 are formed in a cylindrical shape and the container cap unit 10 has a guiding rail(s) 12 on the inner surface thereof so that the container cap unit 10 can be screwably engaged with the container, the shape of the container cap unit 10 (as well as the container) and the microneedle unit 20 and the manner of the engagement thereof with the container shown in FIG. 2 are only for illustration and are thus not limited thereto. For example, the container cap unit 10 and the microneedle unit 20 can be formed in the shape of a quadratic prism and the container cap unit 10 can be engaged with the container in a press-fit manner. Also, although it is shown in FIG. 2 that the microneedle unit 20 and the container cap unit 10 are engaged with each other in a press-fit manner, but the engagement manner is not limited thereto. Further, although it is shown in FIG. 2 that the system has two of the first through-holes 11 and two of the second through-holes 21, the first and second through-holes 11, 21 are in a cylindrical shape, the number and the shape of the first and second through-holes can be determined properly. For instance, the number of the first through-hole 11 may be the same as or different from that of the second through-hole 21. Also for instance, the shape of the first through-hole 11 may be the same as or different from that of the second through-hole 21.

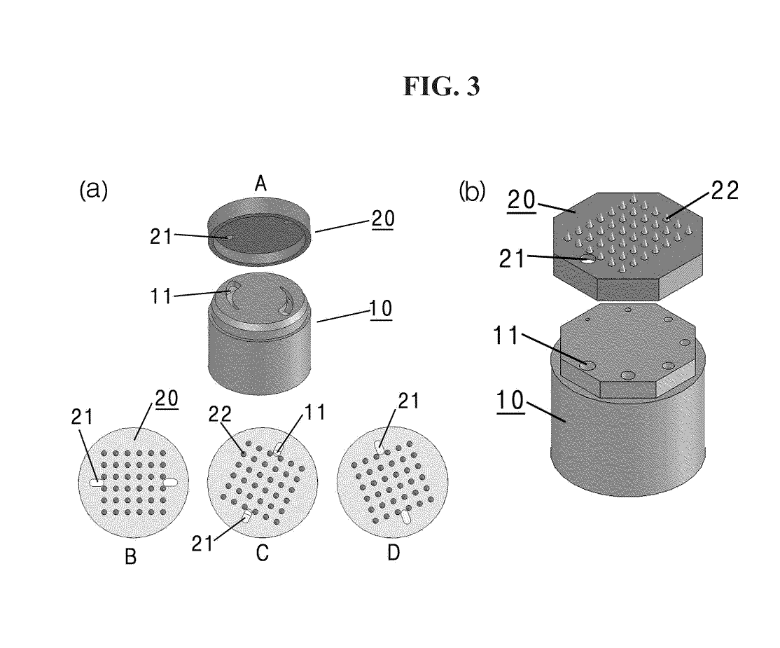

FIG. 3 (a) shows a microneedle cap system according to another embodiment of the present invention, in which the shape of the first through-holes 11 and the shape of the second through-holes 21 are different (A). In this embodiment, each of the first through-holes 11 is formed in the shape of a circumferentially extending arcuate slot on the top surface of the container cap unit 10 in such a fashion that each of the first through-holes 11 is disposed to be equidistantly spaced apart from the central point of the top surface of the container cap unit 10, and one distal end thereof becomes narrower and the other distal end thereof becomes wider.

The second through-holes 21 are formed at positions corresponding to the first through-holes 11. Although it is shown in FIG. 3(a) that the second through-holes 21 are formed in a slit shape, but they are not limited thereto. Preferably, the length (the length in the radial direction from the central point of the top surface of the microneedle unit 20) of the second through-hole 21 may be set to be larger than the width of the narrower distal end of the first through-hole(s) 11. Also preferably, the length of the second through-hole(s) 21 may be set to be equal to or larger than the width of the wider distal end of the first through-hole(s) 11. The lengths of the second through-holes 21 are not limited thereto and can be determined independently and appropriately as long as overlapped area between the first through-hole 11 and the second through-hole 21 can be changed by the relative positions of the container cap unit 10 and the microneedle unit 20.

By changing the relative positions of the container cap unit 10 and the microneedle unit 20, the outflow rate of the active substance can be easily adjusted. In more detail, as shown in FIG. 3(a)B, when the second through-holes 21 are overlapped with the wider distal ends of the first through-holes 11, a greater amount of the active substance may flow out (B). As shown in FIG. 3(a)C, when the second through-holes 21 are overlapped with the narrower distal ends of the first through-holes 11, a smaller amount of the active substance may flow out. As shown in FIG. 3(a)C, when the second through-holes 21 are not overlapped with the first through-holes 11, the active substance may not flow out.

FIG. 3 (b) shows a microneedle cap system according to still another embodiment of the present invention, in which the container cap unit 10 and the outer surface of the upper part of the microneedle unit 20 have the shape of a polygonal prism. In this case, the outer surface of the lower part of the container cap unit 10 and/or the inner surface of the container cap unit 10 may have a different shape from the shape of the outer surface of the upper part of the container cap unit 10. Accordingly, the container (or the upper part of the container) may have a different shape.

In this embodiment, as shown in FIG. 3(b), the microneedle unit 20 has a second through-hole 21. The container cap unit 10 has a plurality of first through-holes 11 such that each of the sides of the polygonal upper surface of the container cap unit 10 has a first through-hole 11. The first through-holes 11 are set to have different sizes. By changing the relative positions of the container cap unit 10 and the microneedle unit 20, overlapped area between the first through-hole 11 and the second through-hole 22 can thus be changed. Although it is shown in FIG. 3(b) that each of the sides of the polygonal upper surface of the container cap unit 10 has a first through-hole 11 and the microneedle unit 20 has a second through-hole 21 and the first and second through-holes 11, 21 have a cylindrical shape, the number and shape of the first and second through-holes 11, 12 may be independently adjusted depending on design needs and/or functional consideration. For example, each of the sides of the polygonal upper surface of the container cap unit 10 may have two, three, or four of the first through-holes 11 and the microneedle unit 20 has two, three, or four of the second through-holes 21 and the first and second through-holes 11, 21 are in the shape of a polygonal prism.

In a modified embodiment of this embodiment, although it is not shown in the drawings, the container cap unit 10 and the outer surface of the upper part of the microneedle unit 20 may be set to have a cylindrical shape, while the first and second through-holes are the ones shown in FIG. 3(b). In a still modified embodiment, the first through-hole 11 may be formed in the shape of a circumferentially extending arcuate slot in such a fashion that the first through-hole 11 is disposed to be equidistantly spaced apart from the central point of the top surface of the container cap unit 10, and one distal end thereof becomes narrower and the other distal end thereof becomes wider, while the first through-hole 11 is the one shown in FIG. 3(a).

The overlapped area between the through-holes of the microneedle unit and the through-holes of the container cap unit can be adjusted depending on the component or use purposes of the active substance so as to adjust the outflow rate of the active substance. If the overlapped area is too large, the active substance can excessively flow out of the container due to erroneous manipulation of the microneedle cap system. The overlapped area may preferably be set not to be greater than a certain value (e.g., 20 mm²).

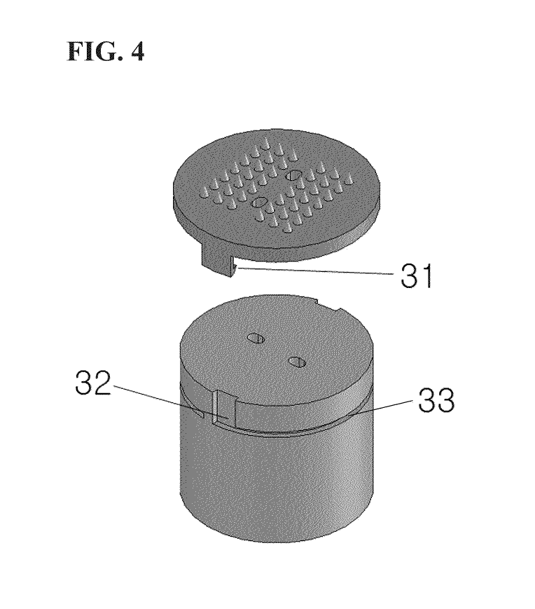

FIG. 4 shows a microneedle cap system according to still yet another embodiment of the present invention. In this embodiment, the microneedle unit 20 has one or more protrusions 31 formed downwardly from the circumferential edge thereof and the container cap unit 10 has one or more recesses 32 formed on the circumferential edge of the top portion thereof. The protrusion(s) 31 can be engaged with the recess(es) in a snap-fit manner so that the microneedle unit 20 is closely fixed to the container cap unit 10, thereby contributing to prevent the active substance from flowing to the outside along the interface (gap) between the container cap unit 10 and the microneedle unit 20.

Preferably, the container cap unit 10 may further include one or more guide grooves 33 formed on the outer circumferential surface thereof. The guide groove(s) 33 circumferentially extend from the recess(es) 32 along the direction of rotation of the protrusions 31 upon the rotation of the microneedle unit 20. The protrusion(s) 31 can be turned in a state of being fitted into the guide groove(s) 33. As a result, when the container unit 10, the microneedle unit 20, or both are turned to adjust the overlapped area between the first through-hole(s) 11 and the second through-hole(s) 21, the container cap unit 10 and the microneedle unit 20 can be maintained in a close contact state.

Although it is shown in FIG. 4 that the guide groove 33 is circumferentially formed on the outer circumferential surface of the container cap unit 10, it is not limited thereto. Further, the protrusion(s) 31 may be formed on the container cap unit 10 and the recess(es) 32 and the guide groove(s) 33 may be formed on the microneedle unit 20. Detailed explanation thereof is omitted since those skilled in the art can readily conceive a modified design to perform the above function.

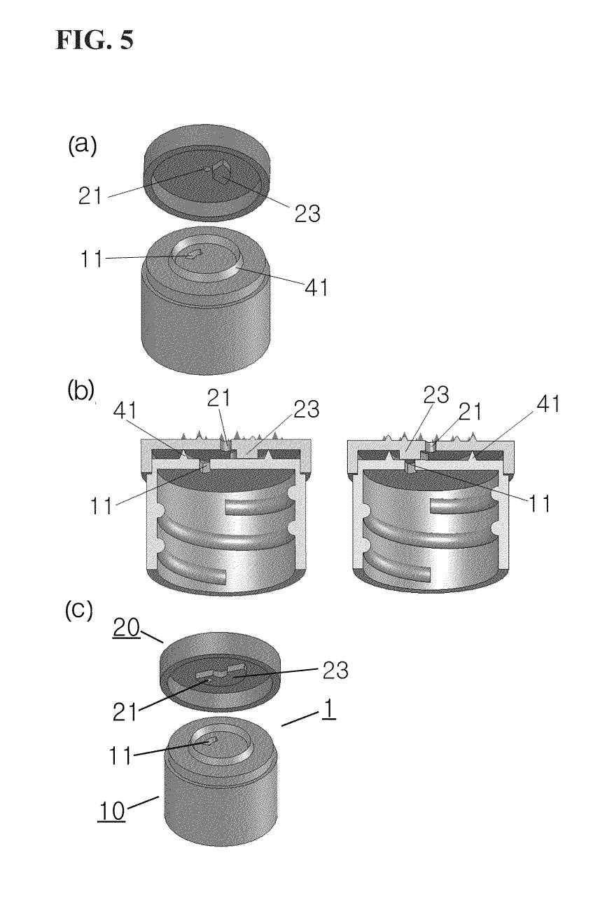

In certain embodiments, as shown in FIGS. 5(a) to 5(c), the container cap unit 10 may suitably have a projecting ring 41 formed on the top surface thereof for preventing the outflow of the active substance, thereby further preventing leakage of the active substance through the interface between the microneedle unit 20 and the container cap unit 10. Although it is shown in FIG. 5 that the projecting ring 41 is formed on the top surface of the container cap unit 10, it may be formed on the bottom surface of the microneedle unit 20 or it may be formed both on the top surface of the container cap unit 10 and on the bottom surface of the microneedle unit 20. The height of the projecting ring can be changed depending on the design needs or functional consideration. Although the material of the projecting ring is not limited to particular ones, it may, preferably, be made of a flexible material such as rubber, silicon or the like.

In the certain embodiments, a stopper protrusion 23 is formed on the bottom surface of the microneedle unit 20. The stopper protrusion 23 is formed to be able to cover entire area of the first through-hole 11. The stopper protrusion 23 can be overlapped with the first through-hole 11 depending on the relative positions between the container cap unit 10 and the microneedle unit 20. As discussed above, by changing the relative positions between the container cap unit 10 and the microneedle unit 20, the degree to which the first through-hole 11 and the stopper protrusion 23 are overlapped with each other can be changed, thereby being able to adjust the outflow rate of the active substance.

In the certain embodiments, as shown in FIG. 5(a), the second through-hole 21 is formed in the microneedle unit 20. As shown in the left side of FIG. 5 (b), when the stopper protrusion 23 is positioned so as not to be overlapped with the first through-hole 11, the active substance may flow out of the container through the first through-hole 11 and then is delivered to the skin through the second through-hole 21. On the other hand, as shown in the right side of FIG. 5 (b), when the stopper protrusion 23 is positioned so as to be overlapped with the first through-hole 11, the active substance may not flow. In modified embodiments, as shown in FIG. 5(c), the second through-hole 21 may be formed in both the stopper protrusion 23 and the microneedle unit 20.

In other modified embodiments, although not shown in the drawings, the stopper protrusion 23 may be formed on the top surface of the container cap unit 10, instead of on the bottom surface of the microneedle unit 20, to be able to be overlapped with the second through-hole 11. In still other modified embodiments, although not show in the drawings, a stopper protrusion 23 may be formed on the bottom surface of the microneedle unit 20 to be able to be overlapped with the first through-hole 11 and another stopper protrusion 23 may be formed on the top surface of the container cap unit 10 to be able to be overlapped with the second through-hole 21.

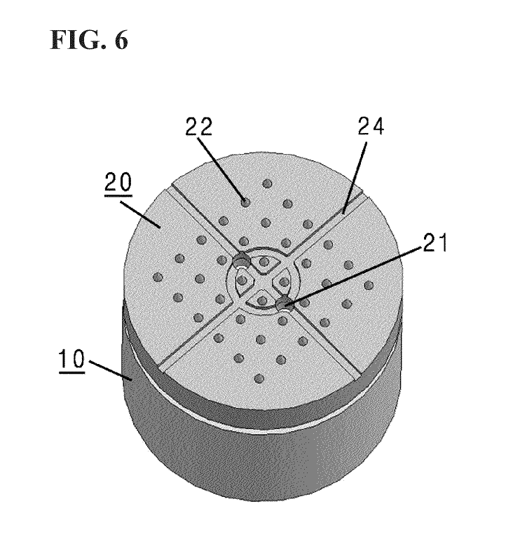

In certain embodiments, as shown in FIG. 6, the microneedle unit 20 may preferably have one or more guide grooves 24 formed on the top surface thereof to guide the flow of the active substance therethrough. Since the active substance flowing along the guide groove(s) 24 can be evenly dispersed among the microneedles of the microneedle array, a desired amount of the active substance can be applied on a desired site of the skin of a subject in a simpler and easier manner.

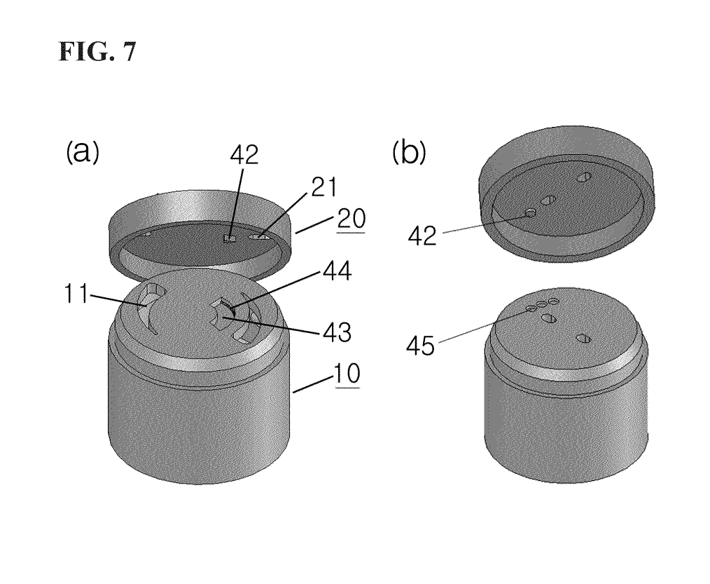

In other certain embodiments, as shown in FIG. 7(a), a rotation adjusting protrusion 42 may be formed on the bottom surface of the microneedle unit 20 and a rotation adjusting resistant serrate section 44 may be formed on the top surface of the container cap unit 10 such that the rotation adjusting protrusion 42 can be moved clickingly through the engagement with the rotation adjusting resistant serrate section 44, thereby allowing the microneedle unit 20 to be rotated in a digital manner. In this case, a rotation recess 43 is preferably formed along an arcuate-shaped path along which the rotation adjusting protrusion 42 is moved. The rotation recess can prevent the leakage of the active substance that may occur due to a space defined between the contact surfaces of the container cap unit 10 and the microneedle unit 20.

Although it is not shown in the drawings, in certain modified embodiments, the rotation adjusting resistant serrate section 44 may be formed on the bottom surface or the centripetal surface of the rotation recess 43. In other words, the rotation adjusting resistant serrate section 44 may be formed on at least one of the bottom surface, the centrifugal surface, and the centripetal surface of the rotation recess 43.

Also, in certain modified embodiments, although not shown in the drawings, the recess 43 and the rotation adjusting resistant serrate section 44 may be formed on the bottom surface of the microneedle unit 20 and the rotation adjusting protrusion 42 may be formed on the top surface of the container cap unit 10.

In certain embodiments, as shown in FIG. 7 (b), a plurality of adjacent rotation adjusting recesses 45, instead of the recess 43 and the rotation adjusting resistant serrate section 44, may be formed on the top surface of the container cap unit 10 along an arcuate-shaped movement path of the rotation adjusting protrusion 42. The protrusion 42 can be fit into one of the rotation adjusting recesses 45. As a result, in these embodiments, the overlapped area between the first through-hole 11 and the second through-hole 21 can be changed in a digital manner, so that the outflow rate of the active substance can be adjusted in a digital manner.

Also, in certain modified embodiments, although not shown in the drawings, the plurality of adjacent rotation adjusting recesses 45 may be formed on the top surface of the container cap unit 10 and the rotation adjusting protrusion 42 may be formed on the bottom surface of the microneedles unit 20.

Further, in certain modified embodiments, the rotation adjusting protrusion 42 may be formed on the inner circumferential surface of the microneedle unit 20 and the recess 43 coupled with rotation adjusting resistant serrate section 44 or the rotation adjusting recesses 45 may be formed on the outer circumferential surface of container cap unit 10. In addition, in still certain modified embodiments, the rotation adjusting protrusion 42 may be formed on the outer circumferential surface of container cap unit 10 and the recess 43 coupled with rotation adjusting resistant serrate section 44 or the rotation adjusting recesses 45 may be formed on the inner circumferential surface of the microneedle unit 20.

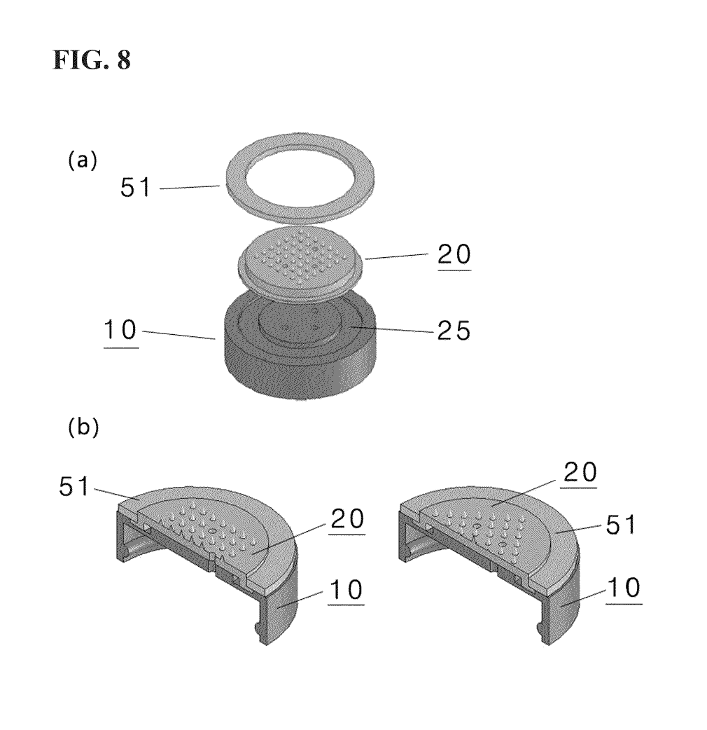

As discussed above, although it is described with respect to the above-described embodiments that the container cap unit 10 and the microneedle unit 20 are engaged with each other in a press-fit manner, the engagement manner is not limited thereto. As an example, the microneedle unit 20 and the container cap unit 10 can be engaged with each other by means of an annular engagement aiding member 51 having a through-opening defined at the center thereof, as illustrated in FIG. 8. In this case, preferably, the container cap unit 10 may have an annular recess 25 formed on the top surface thereof. The annular recess 25, preferably, has the same size as or a smaller size than the microneedle unit 20 so that the microneedle unit 20 can be more easily mounted on the recess 25.

For instance, the container cap unit 10 and the microneedle unit 20 can be engaged by stacking sequentially the container cap unit 10, the microneedle unit 20 which has a size smaller than that of the container cap unit 10, and the engagement aiding member 51 the outer diameter of which is equal to or greater than that of container cap unit 10 and the inner diameter of which is the same as or smaller than the outer diameter of the microneedle unit 20. The region of the bottom surface of the engagement aiding member 51 that is not overlapped with the top surface of the microneedle unit 20 can then be bonded to the top surface of the container cap unit 10. Alternatively, similar to a method of engaging a cap with an injectable agent container, the region (protruding region) of the bottom surface of the engagement aiding member 51 that is not overlapped with the top surface of the microneedle unit 20 can then be compressingly bonded. However, as discussed above, this is an exemplary method of engaging the container cap unit and the microneedle unit 20, and the engagement method according to the present invention is not limited thereto.

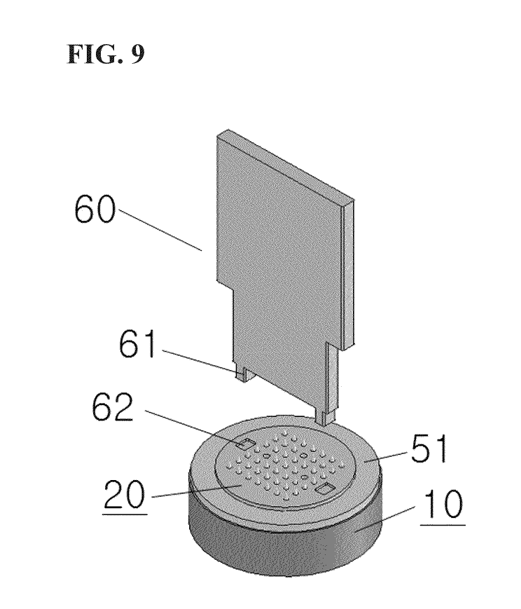

Microneedle cap system according to certain embodiments may be provided with a rotation aiding element 60 for facilitating the change of the relative positions of the microneedle unit 20 with respect to the container cap unit 10, as shown in FIG. 9. In these embodiments, the microneedle unit 20 has one or more rotation aiding recesses 62 formed on the top surface thereof, and the rotation aiding element 60 has one or more protruding pieces 61 formed the bottom end thereof such that the protruding pieces 61 can fit into the rotation aiding recesses 62. The microneedle unit 20 can be easily turned by using the rotation aiding element 60.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A microneedle cap system for being mounted to a container containing an active substance to be delivered to a subject, the microneedle cap system comprising:
a container cap unit configured to be engaged with the container and having one or more first through-holes formed on the top surface thereof through which the active substance contained in the container can pass; and
a microneedle unit configured to be rotatably or detachably engaged with the container cap unit and having a microneedle array 22 formed on the top surface thereof and one or more second through-holes formed therein,
wherein the first through-hole or through-holes can be overlapped with the second through-hole or through-holes depending on the relative position of the microneedle unit with respect to the container cap unit and the area of the region overlapped can be changed by changing the relative position of the microneedle unit with respect to the container cap unit.

2. The microneedle cap system according to claim 1, wherein the first through-hole or through-holes are formed in the shape of a circumferentially extending arcuate slot on the top surface of the container cap unit in such a fashion that the first through-hole or through-hole are disposed to be spaced apart from the central point of the top surface of the container cap unit, and one distal end thereof becomes narrower and other distal end thereof becomes wider.

3. The microneedle cap system according to claim 1, wherein the maximum overlapped area between the second through-hole 21 and the first through-hole 11 is not greater than 20 mm².

4. The microneedle cap system according to claim 1, wherein one of the container cap unit and the microneedle unit has one or more recesses formed thereon, and
wherein the other of the container cap unit and the microneedle unit has one or more protrusions formed thereon that can be fitted into the recesses, and one or more guide grooves formed on the outer circumferential surface thereof and extending from the recesses along the direction of rotation of the protrusions upon the rotation of the microneedle unit.

5. The microneedle cap system according to claim 1, wherein at least one projecting ring is formed on the top surface of the container cap unit or the bottom surface of the microneedle unit for preventing outflow of the active substance,
wherein at least one stopper protrusion is formed on the bottom surface of the container cap unit, on the top surface of the microneedle unit, or on both for covering the first through-hole or through-holes, the second through-hole or through-holes, or both.

6. The microneedle cap system according to claim 5, wherein the projecting ring is made of rubber or silicon.

7. The microneedle cap system according to claim 1, wherein at least one guide groove for guiding the active substance is formed on the top surface of the microneedle unit.

8. The microneedle cap system according to claim 1, wherein at least one rotation adjusting protrusion is formed on one of the container cap unit and the microneedle unit, and
wherein at least one rotation recess formed along an arcuate-shaped path along which the rotation adjusting protrusion can be rotated in response to rotation of the microneedle unit and at least one rotation adjusting resistant serrate section 44 formed on at least one of the centripetal side surface, the centrifugal side surface, and the bottom surface of the rotation recess are formed on the other of the container cap unit and the microneedle unit.

9. The microneedle cap system according to claim 1, wherein at least one rotation adjusting protrusion is formed on one of the container cap unit and the microneedle unit, and
wherein a plurality of adjacent rotation adjusting recesses formed along an arcuate-shaped path along which the rotation adjusting protrusion can be rotated in response to rotation of the microneedle unit are formed on the other of the container cap unit and the microneedle unit.

10. The microneedle cap system according to claim 1, wherein the microneedle unit and the container cap unit are engaged with each other by an engagement aiding member having a through-opening defined at the center thereof.

11. The microneedle cap system according to claim 10, wherein the container cap unit has a recess formed on the top surface thereof, the recess having a size that is the same as or greater than that of the microneedle unit.

12. The microneedle cap system according to claim 1, further including a rotation aiding element which is provided with a protruding piece formed on the bottom end thereof,
wherein the microneedle unit has at least one rotation aiding recess formed on the top surface thereof.
